(19) United States
(12) Patent Application Publication
(10) Pub. No.: US 2011/0123985 A1
(43) Pub. Date: May 26, 2011

(54) COLUMN ENRICHMENT OF PCR BEADS COMPRISING TETHERED AMPLICONS

(75) Inventors: Aldrich N. K. LAU, Palo Alto, CA (US); Achim KARGER, Foster City, CA (US); Patrick KINNEY, Hayward, CA (US); James NURSE, Pleasanton, CA (US); Aftab KEVAL, Hayward, CA (US); Douglas GREINER, Fremont, CA (US); Carmen GJERSTAD, Millbrae, CA (US)

(73) Assignee: APPLIED BIOSYSTEMS, LLC, Carlsbad, CA (US)

(21) Appl. No.: 12/756,589

(22) Filed: Apr. 8, 2010

Related U.S. Application Data
(60) Provisional application No. 61/307,428, filed on Feb. 23, 2010.
(60) Provisional application No. 61/167,781, filed on Apr. 8, 2009.
(60) Provisional application No. 61/167,766, filed on Apr. 8, 2009.

(51) Int. Cl. C12Q 1/68; C12M 1/40

(52) U.S. Cl. 435/6; 435/287.2

(57) ABSTRACT

An enrichment module and method are provided for enriching a population of templated beads and separating them from non-templated beads. The method can include hybridizing a templated bead with an enrichment bead to form a complex, trapping the complex in a filtration medium, washing non-templated beads through the filtration medium while retaining the complex, and then eluting the templated bead from the complex. The module can include a column for enrichment and filtration material exhibiting desired size-exclusion properties.

### CROSS-REFERENCE TO RELATED APPLICATIONS

The present application claims the benefit of the earlier filing date of U.S. Provisional Patent Applications Nos. 61/307,428, filed Feb. 23, 2010, 61/167,781, filed Apr. 8, 2009, and 61/167,766, filed Apr. 8, 2009, each of which is incorporated herein in its entirety by reference.

### FIELD

The present teachings relate to devices, systems, and methods for preparing templated DNA beads.

### INTRODUCTION

A number of biological sample analysis methods rely on sample preparation steps as a precursor to carrying out the analysis methods. For example, a precursor to performing many biological sequencing techniques (e.g., sequencing of nucleic acid) includes amplification of nucleic acid templates in order to obtain a large number of copies (e.g., millions of copies) of the same template.

Polymerase chain reaction is a well understood technique for amplifying nucleic acids which is routinely used to generate sufficiently large DNA populations suitable for downstream analysis. Recently, PCR-based methods have been adapted to amplifying samples contained within emulsions for sequencing applications. In such amplification methods a plurality of biological samples (e.g. nucleic acid samples) may be individually encapsulated in microcapsules of an emulsion and PCR amplification conducted on each of the plurality of encapsulated nucleic acid samples simultaneously. Such microcapsules are often referred to as “microreactors” since the amplification reaction occurs within the microcapsule.

In some cases, the microcapsule may include an enrichment bead and the amplification process may be referred to as bead-based emulsion amplification. In such a technique, beads along with DNA templates are suspended in an aqueous reaction mixture and then encapsulated in a water-in-oil emulsion. The template DNA may be either bound to the bead prior to emulsification or may be included in solution in the amplification reaction mixture. For further details regarding techniques for bead emulsion amplification, reference is made to PCT publication WO 2005/073410 A2, entitled “NUCLEIC ACID AMPLIFICATION WITH CONTINUOUS FLOW EMULSION,” which published internationally on Aug. 11, 2005, and is incorporated by reference in its entirety herein.

A need exists for a method and system for enriching templated beads from a mixture that includes non-templated beads.

### SUMMARY

According to various embodiments, a method is provided for enriching templated beads from a mixture of templated beads and non-templated beads. The method comprises providing a mixture of templated beads and non-templated beads, and combining the mixture with a plurality of enrichment beads. The method can comprise binding one or more of the enrichment beads with one or more of the templated beads to form one or more respective capture complexes. The one or more capture complexes can then be separated from the non-templated beads to form one or more separated capture complexes. In some embodiments, the one or more templated beads can then be separated from the one or more separated capture complexes to form one or more recovered templated beads. The binding can comprise hybridizing the one or more enrichment beads to one or more respective templated beads. The method can further comprise forming the templated beads in an emulsion PCR reaction. The templated beads can comprise PCR-amplicon bearing microspheres.

In some embodiments, separating can comprise using a filtration-based method to selectively isolate templated beads from non-templated beads. In various embodiments, separation may be accomplished using a size-exclusion material. The separating can comprise depth-filtration separation. The recovering can comprise eluting one or more of the templated beads from the one or more separated capture complexes. The recovering can comprise passing the templated beads through the filter and retaining the enrichment beads. Each of the templated beads and each of the non-templated beads can have a diameter of from 0.1 μm to 1.2 μm, from 0.25 μm to 2.0 μm, from 0.2 μm to 1.0 μm, from 0.3 μm to 0.9 μm, or from 0.7 μm to 1.1 μm. The one or more enrichment beads can each have a diameter, or collectively an average diameter, of from 3.0 μm to 20 μm, for example, from 5.0 μm to 15 μm, or from about 6.4 μm to about 6.8 μm. In some embodiments, the method can further comprise denaturing a template or amplicon tethered to the one or more recovered templated bead. In some embodiments, the templated beads and the non-templated beads can each comprise a metal material or a ferromagnetic material and the method can further comprise magnetically manipulating the recovered templated beads, for example, to arrange them on a slide or within a flowcell. The method can further comprise carrying out sequencing reactions on the beads so arranged.

According to various embodiments, a system is provided for the enrichment of templated beads from a mixture of templated beads and non-templated beads. The system can comprise a mixture of templated beads and non-templated beads having a first average diameter, a plurality of enrichment beads having a second diameter, and a separation device comprising a size-exclusion filtration material having an average pore size. Each enrichment bead can be functionalized to bind with one or more of the templated beads to form one or more respective capture complexes. The average pore size can be larger than the first average diameter and smaller than the second diameter. The filtration material can comprise a hydrophobic material. The filtration material can comprise a polypropylene material. The templated beads can comprise PCR-amplicon bearing microspheres. The PCR-amplicon bearing microspheres can comprise respective clonal populations of amplicons. Each enrichment bead can be functionalized to hybridize with one or more of the templated beads. The system can comprise one or more buffer solutions disposed in one or more pre-filled containers. The system can comprise a thermomixer configured to prepare enrichment beads. The system can comprise a dia-filtration column configured to purify and agitate templated beads.

According to various embodiments a system is provided that comprises an emulsifier module, an amplifier module, and an enrichment module, which together can be used to form templated beads useful in a bead-based DNA sequencing platform. In some embodiments, the system can comprise in-line filters to non-magnetically concentrate beads and perform buffer exchanges. In some embodiments, a dia-filtration unit and method can be used in lieu of a manual glycerol cushion and centrifugation. In some embodiments, beads are de-aggregated using sheer flow through a syringe valve.

According to various embodiments, an enrichment module and method are provided for enriching a concentration of templated beads and separating them from non-templated beads. The method can comprise hybridizing a templated bead with an enrichment bead to form a complex, trapping the complex in a filtration medium, washing non-templated beads through the filtration medium while retaining the complex, and then eluting the templated bead from the complex. The module can comprise a column for enrichment and filtration material exhibiting desired size-exclusion properties.

### BRIEF DESCRIPTION OF THE DRAWINGS

The accompanying drawings, which are incorporated in and constitute a part of this specification, illustrate exemplary embodiments and together with the description, serve to explain various principles. The skilled artisan will understand that the drawings, described below, are for illustration purposes only. The drawings are not intended to limit the scope of the present teachings in any way.

FIG. 1 shows a templated bead workflow from emulsion generation to bead enrichment, according to various embodiments of the present teachings.

FIG. 2 is a flowchart showing exemplary process steps that can be carried out by a method and system according to various embodiments of the present teachings.

FIGS. 3A-3C show load, wash, and elute steps, respectively, according to various embodiments of the present teachings.

FIG. 4 shows a polypropylene prefilter material comprising hydrophobic 2.5 μm material that can be used according to various embodiments of the present teachings.

FIG. 5 shows an enrichment module according to various embodiments of the present teachings.

FIG. 6 is an enlarged view of the center deck portion of the enrichment module shown in FIG. 5

### DESCRIPTION

According to various embodiments of the present teachings, an emulsion is created that comprises droplets of an aqueous phase, or microreactors, in which clonal amplification takes place. Microreactors containing a single template bead and a single template, called monoclonal microreactors, are desired and can be formed according to the present teachings. Some microreactors, however, can be polyclonal such that they contain multiple templates, non-clonal such that they contain no template, or multi-bead-containing, and some microreactors exhibit a combination of these features.

After the emulsion is created, it can be thermally cycled to produce, for example, more than 30,000 copies of template amplified on to each template bead. Each template bead can comprise a respective primer, for example, a P1 primer, attached to a bead. In non-clonal microreactors, the template bead cannot amplify. Although beads are referred to often herein, it is to be understood that other template or target supports can be used, for example, particles, granules, rods, spheres, shells, combinations thereof, and the like. Furthermore, although the microreactors are described herein as containing components for PCR, it is to be understood that the microreactors can contain components for reactions other than PCR, for example, components for an isothermal reactions, components for another amplification reaction, components for an enzymatic reaction, components for a ligation reaction, or the like.

After emulsion PCR is complete, some of the template beads comprise amplicons of the template formed thereon, and are herein referred to as templated beads. Templated beads comprise template beads on which amplification took place in the respective microreactors. Some of the template beads do not comprise amplicons of the template formed thereon, and are herein referred to as non-templated beads. Non-templated beads comprise template beads on which no amplification took place in the respective microreactors. The non-templated beads can also be referred to as non-amplifying beads.

The emulsion can then be broken, for example, with 2-butanol, and the templated beads and non-templated beads can be recovered and washed. Enrichment can be performed to isolate template beads from non-templated beads. In some embodiments, an enrichment bead comprising a single-stranded P2 adaptor or P2 primer can be used to capture the templated beads. The mixture of enrichment beads, enrichment bead-templated bead complexes, and non-templated beads, can then be subject to filtration followed by elution to isolate the templated beads.

In some embodiments, each of the templated beads and each of the non-templated beads can have a diameter of from 0.25 μm to 2.0 μm, from 0.5 μm to 1.0 μm, from 0.9 μm to 1.2 μm, or from 0.7 μm to 1.1 μm. In some embodiments, the one or more enrichment beads can each have a diameter, or collectively an average diameter, of from 3.0 μm to 20 μm, for example, from 5.0 μm to 15 μm, from 6.0 μm to 10 μm, or from 6.4 μm to 6.8 μm.

Reference will now be made in detail to various exemplary embodiments, examples of which are illustrated in the accompanying drawings. Wherever possible, the same reference numbers will be used throughout the drawings to refer to the same or like parts.

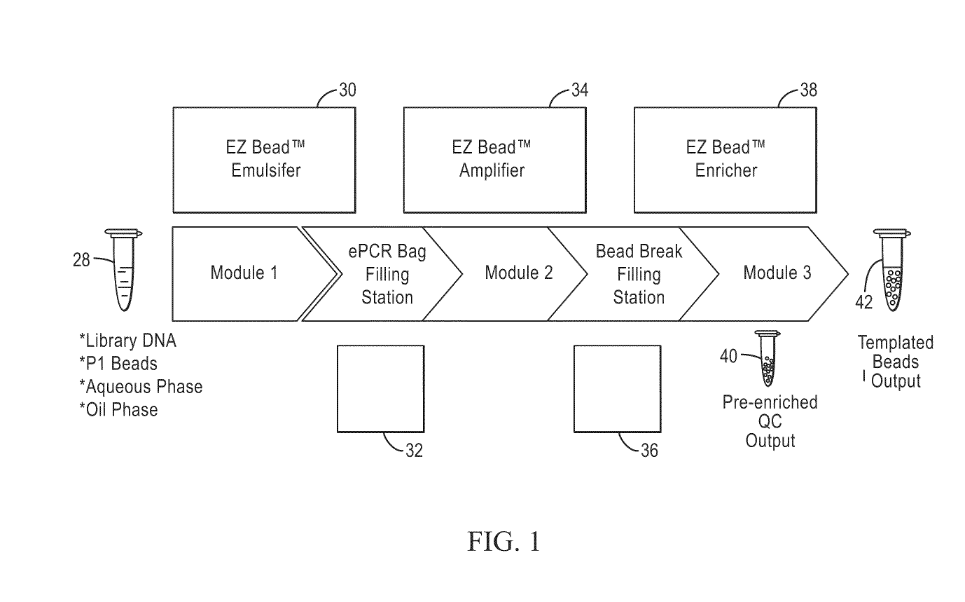

FIG. 1 shows a templated bead workflow from emulsion generation to bead enrichment, according to various embodiments of the present teachings. FIG. 1 shows an exemplary process workflow and the system components for carrying out the process. An input sample 28 to be processed by the system can comprise an aqueous phase component such as a master mix, an oil phase component such as a master mix, template beads, and a collection or library of templates such as DNA sample molecules from the same or from different samples. The aqueous phase master mix can comprise water, dNTPs, buffers, salt, and DNA polymerase. The various components for the emulsion can be brought together and emulsified in an emulsifier module 30 during a first step of the multi-step process depicted. Emulsifier module 30 is also referred to herein as module 1 in the process flow diagram shown in FIG. 1. The emulsion can be made by conventional techniques in some embodiments. After forming an emulsion using emulsifier module 30, the mixture can be poured into a pouch using an ePCR pouch filling station 32. The pouch can comprise a bag or other flexible container. After filling, the pouch can be closed or sealed, for example, by heat-sealing. The pouch and its contents can then be thermally cycled using an amplification module 34 that is also referred to herein as module 2 in the process flow diagram shown in FIG. 1. The amplification module can serve to amplify template molecules, for example, by thermal cycling. Following amplification sing amplification module 34, the contents of the pouch can be poured into a break vessel at a break filling station 36 to carry out a fourth step of the multi-step process.

After breaking the emulsion to release the beads, the templated beads can be enriched using an enrichment module 38 that is also referred to herein as module 3 in the process flow diagram shown in FIG. 1. The beads can comprise productive beads, referred to herein as templated beads. Templated beads can comprise beads that have undergone a desired reaction, for example, upon the surface of which multiple reactions have taken place. The beads can also comprise non-templated beads, which were not productive.

According to various embodiments, there can be two or more outputs of the system, including, for example, a first output 40 that includes a pre-enriched quality control output that can provide a user with information on bead clonality, for example, yield, purity, concentration, and the like. A second output 42 can be provided that includes templated beads that are ready for further processing such as terminal transferase modification, deposition on a slide or in a flow cell, a combination thereof, or the like.

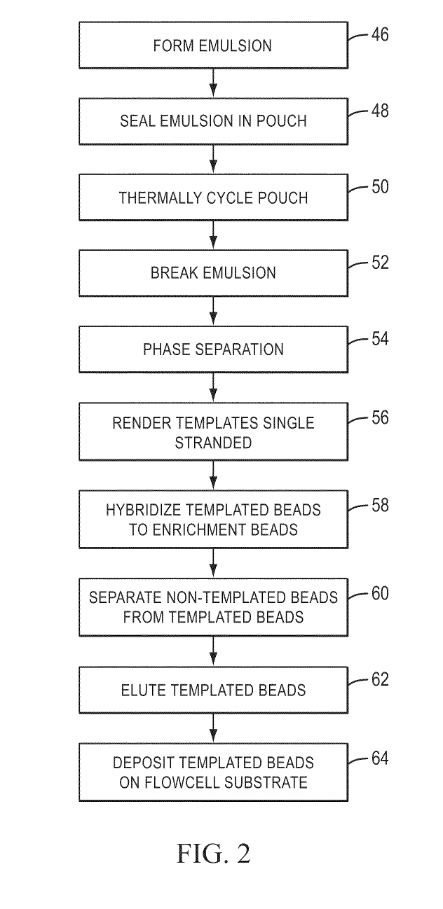

While the system described in connection with FIG. 1 comprises various different modules and stations, and process steps, it is to be understood that the system can comprise less or more modules and/or stations and that various modules and/or stations can be combined together. Furthermore, it is to be understood that the method can comprise fewer or more steps than the exemplary steps described in connection with FIG. 1 can each independently be omitted or combined with one or more other steps. In some embodiments, other amplification reactions, isothermal amplification reactions, enzymatic reactions, biological reactions, and the like, can be carried out instead of or in addition to a polymerase chain reaction. Moreover, additional steps can be provided in the method as exemplified with reference to FIG. 2.

FIG. 2 is a process flow diagram showing various process steps associated with a method according to various embodiments of the present teachings. As with FIG. 1, the process steps shown in FIG. 2 can each independently be omitted, substituted, or combined with one or more other process steps. As shown in FIG. 2, a first step 46 of the method can comprise forming an emulsion. The emulsion can be formed according to any of the various embodiments of the present teachings and as described herein. In a next step 48, the emulsion is sealed in a pouch. In an exemplary embodiment, the pouch can comprise a heat-sealable material and the sealing can comprise heat sealing the emulsion in the pouch. The sealed pouch can then be thermally cycled as depicted by process step 50.

In an exemplary embodiment, a dual-sided thermal cycler is used to amplify the emulsion in the pouch. The amplification can result in templated beads each comprising amplicons of a respective template tethered or hybridized to a primer pre-deposited on a surface of a respective template bead. The method can further comprise an emulsion breaking step 52 followed by a phase separation step 54, tailored to separate and/or purify the templated beads from the remainder of the emulsion. A denaturing step 56 can be provided to render the templates tethered to the templated beads, single stranded.

Templated beads bearing the single-stranded templates can be hybridized to enrichment beads to form a capture complex, as depicted at step 58 and described in more detail below. In the next step, the templated beads captured in the capture complexes can be separated from non-templated beads in a separation step 60, for example, using a size-exclusion technique. In a next step 62, the productive or templated beads are separated or eluted from the capture complexes and are collected. Subsequently, the collected productive or templated beads can be made ready for other operations including, for example, deposition on a flow cell substrate or otherwise formed into an array in a flow cell.

According to various embodiments, the emulsion can be formed by mixing together an aqueous phase solution, a plurality of template-capturing beads, a collection or library of sample templates or nucleic acid fragments, DNA polymerase, other enzymes, buffers, salts, and a pair of primers, to form a mixture. The mixture can then be contacted with an oil phase and emulsified to form an emulsion comprising a plurality of microreactors. On exemplary approach to emulsification is described, for example, in concurrently filed U.S. patent application Ser. No. ______ to Lau et al., entitled “System and Method for Preparing and Using Bulk Emulsion,” Attorney Docket No. 5010-480-01, which is incorporated herein in its entirety by reference.

According to various embodiments, the emulsion can comprise an aqueous phase and an oil phase wherein the aqueous phase comprises components useful for a desired reaction, for example, components for amplifying DNA templates such as a library of templates from a single sample. In some embodiments, the emulsion comprises clonal or monoclonal reactors or microreactors containing a single DNA template molecule. Some sequencing platforms, for example, the SOLiD sequencing system by Applied Biosystems, Foster City, Calif., utilize emulsion polymerase chain reaction (ePCR) approaches that provide compartmentalization of PCR reactions in discrete aqueous droplets of an inverse emulsion such as a water-in-oil (W/O) emulsion. In some embodiments, a template bead, approximately 1 μm in diameter, and comprising surface-immobilized oligo nucleotides, can be entrapped in each discrete aqueous droplet microreactor. Each microreactor can also contain PCR reagents such as a forward primer, a reverse primer, a DNA polymerase, and a single DNA sample molecule.

In some cases, some of the microreactors can comprise some of the components but not others. For example, some microreactors can contain no template and no DNA polymerase, and would not be expected to yield a templated bead. According to various embodiments, the microreactors can contain other components for reactions other than PCR, for example, components for an isothermal amplification, components for another amplification reaction, components for an enzymatic reaction, components for a ligation reaction, or the like.

In various embodiments, the emulsion is thermally cycled from approximately 64° C. to 96° C. for 40 or 60 cycles (depending on the length of the template molecule being used). Subjecting the microreactors to PCR conditions in this manner results in clonal amplification yielding a product that is composed of a singular DNA species. The amplification conditions can cause a templated bead to be formed in many of the microreactors. Concentrations of components can be used to minimize the number of microreactors containing two or more templated beads. The microreactors can include microreactors that contain no template molecule or no template bead and thus do not produce a templated bead.

The emulsion preparation system and method can be adapted to readily prepare a wide range of different emulsion volumes, for example, of from approximately 5 mL to 250 mL or more, without maintaining a stock of differently sized or configured consumables to accommodate a particular emulsion volume. The emulsion exhibit small drop size variation, a slow rate of reversion or phase separation, and an adaptability to a wide variety of volume sizes. Additionally, the emulsion preparation apparatus of the present teachings is cost-effective, user-friendly, and robust, and provides a reproducible means to prepare inverse emulsions for ePCR.

In some embodiments, the present teachings provide devices, methods, and formulations for the preparation of inverse (water-in-oil) emulsions for polymerase chain reactions. In various embodiments, the discrete aqueous phase (droplets) can entrap a particle, for example, a magnetic particle of about 1 μm diameter size and having oligonucleotides such as one or more different types of primers immobilized on its surface. The discrete aqueous phase droplet can also comprise PCR reagents such as dNTPs, enzymes, co-enzymes, salts, buffers, surfactants, and a template molecule such as a DNA sample. The template molecule can be a sample DNA molecule, for example, a template from a library of templates from a single sample. The continuous phase can comprise oil with or without added surfactants that have hydrophilic-lipophilic-balances (HLB) values equal to or less than 5.0 and below. According to various embodiments of the invention, the surfactants can comprise a mixture of surfactants having various HLB values. Those who are skilled in the art can appreciate that the surfactant affinity different (SAD) of an oil phase can be adjusted by using various surfactants with various HLB values such that a stable inverse (water-in-oil) emulsion can be prepared.

The liquid oil phase can comprise a mineral oil such as Petroleum Special, an alkane such as heptadecane, a halogenated alkane such as bromohexadecane, an alkylarene, a halogenated alkyarene, an ether, or an ester having a boiling temperature above 100° C. The oil phase can be insoluble or slightly soluble in water. The ratio between the continuous oil phase and the discrete aqueous phase may range from 1/0.1 v/v to 4/1 v/v, from 0.5/1 to 3/1, from 0.8/1 to 1/1, or as desired.

According to various embodiments, the emulsion can be placed in a sealed pouch and the sealed pouch can be placed in a dual-sided amplifier or thermocycler. The emulsion in the pouch can be reacted or thermally cycled. The emulsion in the pouch can be subjected to a reaction, for example, an enzymatic reaction such as a polymerase chain reaction, using a thermal cycler and method as described, for example, in concurrently filed U.S. patent application Ser. No. ______ to Liu et al., entitled “System Comprising Dual-Sided Thermal Cycler and Emulsion PCR in Pouch,” Attorney Docket No. 5010-480-02, which is incorporated herein in its entirety by reference. After amplification, the amplified products are then subjected to subsequent downstream processing, including emulsion breaking, bead enrichment, array deposition of beads, and sequencing.

According to various embodiments, a method is provided for enriching templated beads from a mixture of templated beads and non-templated beads. The method comprises providing a mixture of templated beads and non-templated beads, and combining the mixture with a plurality of enrichment beads. The method can comprise binding one or more of the enrichment beads with one or more of the templated beads to form one or more respective capture complexes. The one or more capture complexes can then be separated from the non-templated beads to form one or more separated capture complexes. In some embodiments, the one or more templated beads can then be separated from the one or more separated capture complexes to form one or more recovered templated beads. The binding can comprise affinity capture or hybridizing the one or more enrichment beads to one or more respective templated beads. The method can further comprise forming the templated beads in an emulsion PCR reaction. The templated beads can comprise PCR-amplicon bearing microspheres.

Conventional separation techniques such as glycerol cushioning, to separate templated beads from non-templated beads, can be labor intensive, provide lower yield, and can be non-amenable to automation. The present teachings overcome such problems.

In some embodiments, separating can comprise using a size-exclusion filtration material or other separation approach. The separating can comprise depth-filtration separation. The recovering can comprise eluting one or more of the templated beads from the one or more separated capture complexes. The recovering can comprise passing the templated beads through the filter and retaining the enrichment beads. Each of the templated beads and each of the non-templated beads can have a diameter of from 0.1 μm to 1.2 μm, from 0.25 μm to 2.0 μm, or from 0.7 μm to 1.1 μm. The one or more enrichment beads can each have a diameter, or collectively an average diameter, of from 3.0 μm to 20 μm, for example, from 6.4 μm to 6.8 μm. In some embodiments, the method can further comprise denaturing a template or amplicon tethered to the one or more recovered templated bead. In some embodiments, the templated beads and the non-templated beads can each comprise a metal and/or ferromagnetic material and the method can further comprise magnetically manipulating the recovered templated beads, for example, to arrange them on a slide or within a flowcell. The method can further comprise carrying out sequencing reactions on the beads so arranged. The filtration based on size-exclusion provides many benefits as the emulsion can be a sticky mess but size-exclusion enables multiple washings, is a quick process, and is suitable to automation.

According to various embodiments, a high load-capacity depth filtration system is provided for the enrichment of PCR-amplicon carrying microspheres from emulsion PCR reactions using bead hybridization capture. Such a system can prevent clogging, can handle large volumes, does not retain the desired product upon elution, and can use a fibrous material as opposed to a through-hole plate. In some embodiments, a through-hole plate or fit is used. The system can provide a way to separate ePCR beads bound to enrichment beads from unbound beads relying on a very robust filtration approach rather than centrifugation or cross-flow filtration. In some embodiments, separation of hybridization captured beads from unbound beads relies on a high load-capacity filter with a nominal pore size allowing ePCR beads to pass and enrichment beads to be retained (ePCR assay bead diameter<filter pore size<enrichment bead diameter).

According to various embodiments, other systems can be provided to separate ePCR beads bound to enrichment beads from unbound beads. In exemplary embodiments, the enrichment beads can be captured based on an affinity-based approach or based on a property specific to them, for example, if modified with streptavidin or biotin the enrichment beads can be captured through a biotin/streptavidin interaction. The enrichment beads or capture complexes can be covalently bound to a support, ionically bound, entangled, entrapped, or the like, without necessarily requiring a size-exclusion technique. In some embodiments, a composite of materials or layers can be used to provide specific size-exclusion properties. In some embodiments, a through-hole plate or frit can be used to separate the beads by size-exclusion.

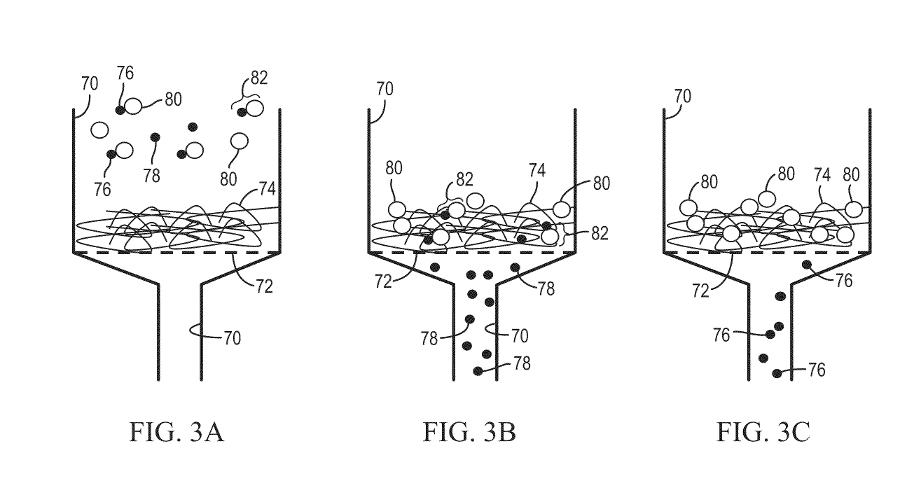

FIGS. 3A-3C show a schematic diagram of a method involving depth filtration and elution of PCR product beads for an ePCR reaction containing an excess of un-reacted ePCR beads. Several rounds of buffer optimization can be used to determine an appropriate hybridization buffer that can be used for filtration. As an example, a buffer comprising 0.1% or more, from 1% to 5%, from 1.5% to 3%, or 2% by volume TWEEN®-20 non-ionic detergent can be used to avoid non-specific excessive adsorption-based binding of un-extended contaminant beads to the filters. In various embodiments, non-specific binding can be evidenced by staining of the filter material with a brownish tint caused by the presence of residual paramagnetic beads.

FIGS. 3A-3C show a load step, a wash step, and an elute step, respectively, according to various embodiments of the present teachings. In the step shown in FIG. 3A, a hybridization buffer is used. In the wash step shown in FIG. 3B, a hybridization buffer is used. In the elute step shown in FIG. 3C, an eluent is used that resets the buffer.

In the step shown in FIG. 3A, a column 70 is used that contains a filter fit 72 or sieve supporting a filtration medium 74. Filtration medium 74 can comprise a polymeric material, for example, a fibrous material that provides size-exclusion properties. Filtration material 74 can be fibrous, granular, porous, or the like, and can be hydrophobic. In some embodiments, filtration material 74 comprises a polyalkylene material, a polyalkylene blend material, a polyalkylene material, or the like. In some embodiments, filtration material 74 can comprise a material that forms pore sizes of from 1 μm to 5 μm, for example, from 1.5 μm to 4 μm, from 2 μm to 3 μm, or about 2.5 μm.

As shown in FIG. 3A, a mixture of templated beads 76, non-templated beads 78, enrichment beads 80, and templated bead complexes 82, is added to column 70 containing filter frit 72 and filtration material 74. The templated beads can each comprise a plurality of amplicons of a single template molecule, and the amplicons can result from PCR amplification of the template in the presence of a DNA bead, that is, a bead to which the template and amplicons can be hybridized. The mixture can be formed, for example, by amplifying an emulsion prepared by first mixing together an aqueous phase solution, a plurality of template-capturing beads, a library of templates from a sample, DNA polymerase, and a pair of primers, to form a mixture, then combining the mixture with an oil phase and emulsifying the resultant combination. For example, the emulsion can be formed as described, for example, in concurrently filed U.S. patent application Ser. No. ______ to Lau et al., entitled “System and Method for Preparing and Using Bulk Emulsion,” Attorney Docket No. 5010-480-01, which is incorporated herein in its entirety by reference.

As shown in FIG. 3A, most of templated beads 76 are captured by and/or bound to enrichment beads 80, for example, through a hybridization reaction. Non-templated beads 78 are not captured by enrichment beads 80. The pore size of the filtration medium can be selected so that templated bead complexes 82 cannot pass through due to their size but non-captured templated beads 76 and non-templated beads 78 can pass through as shown in FIG. 3B, due to their size.

FIG. 3B shows a wash step wherein a wash solution or wash buffer is used to wash the smaller, non-templated beads 78 through filtration medium 74 and filter fit 72 and out of column 70 as well as washing out undesired residual aqueous and oil components. As can be seen, the sizes of non-templated beads 78 are such that they pass through filtration medium 74 whereas the sizes or affinities of templated bead complexes 82 are such that they do not pass through filtration medium 74. Templated beads 76 which are targeted for collection thus remain complexed with enrichment beads 80 in filtration medium 74 in column 70, and do not wash out. Such a size-exclusivity feature can be accomplished, for example, by using a filtration medium 74 having a 2.5 μm pore size, by using templated beads and non-templated beads having a sub-2.5 μm size diameter, and by using enrichment beads 80 having an average size diameter that is greater than 2.5 μm. As will be understood by those of skill in the art, many combinations of sizes can be used to achieve size-exclusivity in this fashion. In some embodiments, multiple washes can be performed, for example, using the same or different wash solutions or buffers each time.

In some embodiments, enrichment beads 80 can comprise polymeric beads, for example, cross-linked polystyrene beads, polystyrene beads, polypropylene beads, or the like. Enrichment beads 80 can comprise an adapter, primer, linkage group, or other functional group tethered or bound to a surface thereof to capture, hybridize, bind, and/or retain templated beads. Templated beads 76 can comprise monoclonal beads, that is, beads to which a single template nucleic acid molecule has been amplified. Other possible “productive” beads can exist, for example, by adapting an approach for beads to which polyclonal nucleic acids are present, or proteins, or peptides, or other desired sample templates. Multiple different templates can be bound to each template bead, in some embodiments, and each can be primed by a different primer.

In some embodiments, no filter or bead pre-treatment, such as passivation of the filter fiber or the beads, for example, with bead block reagent, is used. Pre-wetting can be used if a buffer containing appropriate concentrations (for example, from about 0.1% to 2%) of POLYSORBATE 20 (TWEEN®-20) detergent is included in the buffer. For washing, in some embodiments, a wash step can comprise applying ‘HYB-T’ buffer, and a 1:1 mixture LSBB and TEX (available from Applied Biosystems, Foster City, Calif.) supplemented with a detergent, for example, from 0.1% to 2% volume/volume TWEEN®-20.

In a next step, as shown in FIG. 3C, templated beads 76 which are targeted for collection are eluted from column 70 using an eluent that de-complexes or releases templated beads 76 from enrichment beads 80. Templated beads 76 can then be collected, for example, at an outlet of column 70. After elution, enrichment beads 80 remain in filtration medium 74, and are not eluted by virtue of their size. In some embodiments, multiple elution steps can be performed, for example, using the same or different eluents each time.

Elution of the beads can involve other desirable processing steps including, for example, a denaturation step that can be done by applying Elution buffer to the filter and incubating for 2 minutes followed by the application of an equal volume of Neutralizer buffer and several volumes of Bead Break & Wash buffer, until all ePCR beads have cleared the filter. The elution process can be repeated if desired. Other processing steps can also be used, for example, chemical treating, washing, labeling, and the like.

For depth filtration, 2.5 μm pore size PP pre-filters (Cat # AN2504700, Millipore) and Streptavidin Coated Polystyrene Particles, 0.5% w/v, 6.0-8.0 μm, 5 mL (Spherotech SVP-60-5), can be used. A kit is provided according to various embodiments, comprising these elements and others, for example, including Bead Break & Wash buffer (SOLiD Templated Bead Preparation Kit), TEX buffer (SOLiD Templated Bead Preparation Kit), B&W buffer or bind and wash buffer (SOLiD Templated Bead Preparation Kit), 2% TWEEN®-20, LSBB Low salt binding buffer (SOLiD Templated Bead Preparation Kit), Elution buffer (0.125 M NaOH, 0.1M NaCl), Hybridization buffer (1:1 mix of TEX:LSBB) and 2% Tween®-20, wherein the Solid kit refers to the SOLiD platform kit available from Applied Biosystems of Foster City, Calif.

The schematic drawings in FIGS. 3A-3C illustrate the depth-filtration separation of templated and non-templated ePCR beads for a mixture of particles resulting from ePCR, and subsequent hybridization capture with larger size enrichment beads. An exemplary protocol uses 1 μm diameter size ePCR beads and 3.4 μm diameter (Spherotech), such that, for example, a polypropylene prefilter (Millipore AN2504700) with an appropriate intermediate pore size of 2.5 μm can be used. To enhance the filter cleanup of the sample by filtration technology, 6.7 μm diameter (Spherotech) enrichment beads can be used. The larger size enrichment beads can be used in equal weight amounts (650 μl of 0.5% w/v slurry, titer 30 micro enrichment beads/ml) per hybridization reaction, to bind approximately 200 to 300 micro ePCR beads per enrichment bead. Other sizes can be used and with affinity separation is not necessarily based on size differences.

Mesh filters that can be used in conjunction with the 1 μm diameter ePCR beads and 6.7 μm diameter enrichment beads, can comprise, for example, Spherotech streptavidin coated filters, and filters comprising type A/D glass fiber, for example, having a 3.1 μm nominal pore size coarse (available as Catalog no. 66220, from Pall Corporation). Other materials that can be used include 25 mm GD/X syringe filters GF/D w/GMF, 2.7 μm pore size (Cat # 6888-2527, available from Whatman) and glass microfiber 934-AH, 1.5 μm pore size (Cat # 6892-2515, available from Whatman). In some embodiments, prefilters made of polypropylene mesh, such as 1.2, 2.5, and/or 5.0 μm pore size PP (Cat # AN1204700, AN2504700 and AN5004700, available from Millipore), can be used. In some embodiments, high-loading capacity, depth syringe filters of 5.0 μm pore size PVDF mesh depth filter, Millex SV, (Cat# SLSV025LS, available form Millipore), can be used.

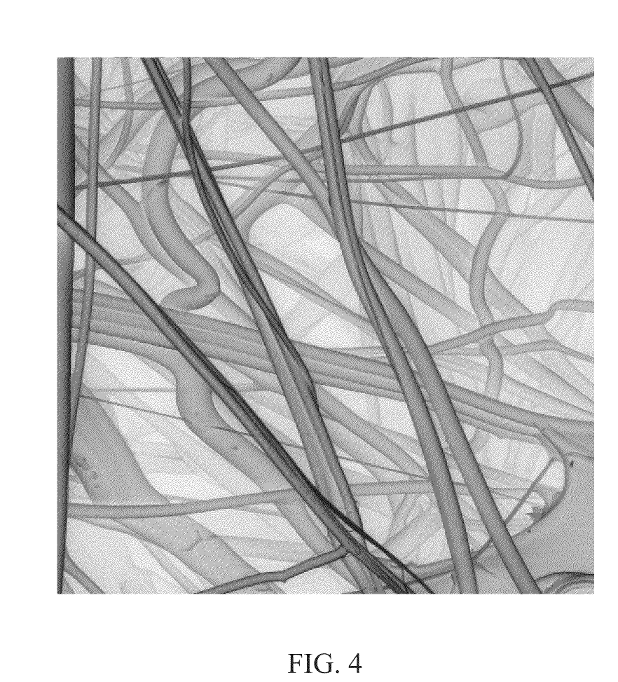

FIG. 4 shows a polypropylene prefilter material comprising hydrophobic 2.5 μm material available from Millipore, can be used as filtration material 74 shown in FIGS. 3A-3C. As can be seen, a fibrous material can be used that forms a resultant porosity that excludes particles of a desired size or larger. The filtration material can comprise a hydrophobic material in some embodiments, which facilitates passing-through of particles smaller than the average pore size.

In some embodiments, the method for the enrichment of ePCR beads: desirably avoids the use of preparation of a glycerol gradient and a centrifugation step; desirably avoids the use of a magnetic concentration step; is independent of magnetic properties of the ePCR beads and can therefore be more generally applied to paramagnetic as well as standard non-magnetic microspheres; can be carried out as a simple bench procedure in the lab; is amenable to automation; uses straight forward wash steps with the help of disposable components; and is an alternative to a glycerol density centrifugation.

According to various embodiments, a system is provided for the enrichment of templated beads from a mixture of templated beads and non-templated beads. The system can comprise a mixture of templated beads and non-templated beads having a first average diameter, a plurality of enrichment beads having a second diameter, and a separation device comprising a size-exclusion filtration material having an average pore size, or another separation device or technique such as using affinity capture. Each enrichment bead can be functionalized to bind with one or more of the templated beads to form one or more respective capture complexes. The average pore size can be larger than the first average diameter and smaller than the second diameter. The filtration material can comprise a hydrophobic material. The filtration material can comprise a chemically inert, non-interacting material such as a polypropylene material. The templated beads can comprise PCR-amplicon bearing microspheres. The PCR-amplicon bearing microspheres can comprise respective monoclonal populations of amplicons. Each enrichment bead can be functionalized to hybridize with one or more of the templated beads. The system can comprise one or more buffer solutions disposed in one or more pre-filled containers. The system can comprise a thermomixer configured to prepare enrichment beads. The system can comprise a dia-filtration column configured to purify and agitate templated beads.

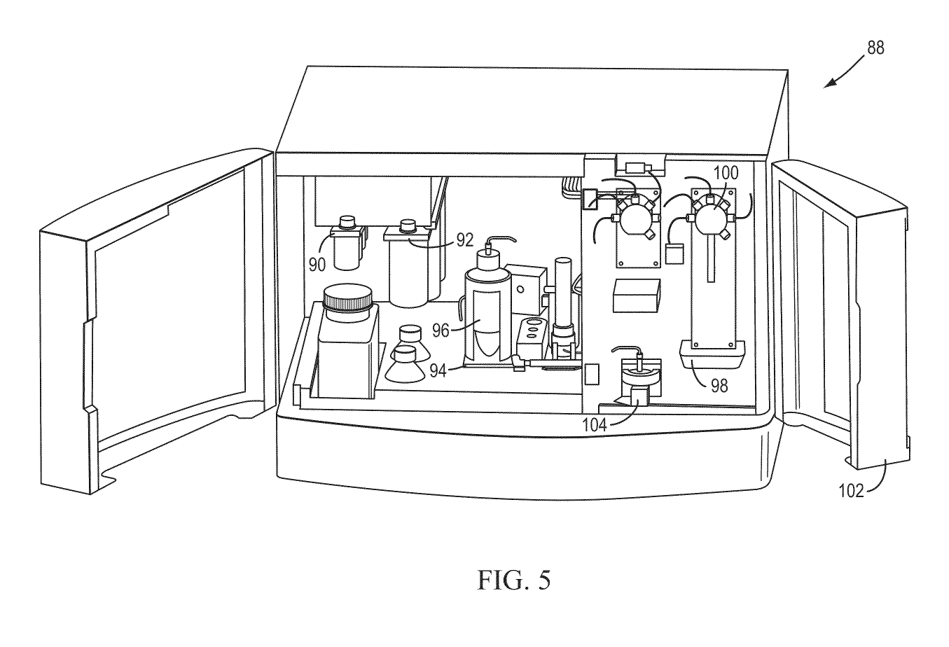

FIG. 5 shows an enrichment module 88 according to various embodiments of the present teachings. On the upper left, are two pre-assembled reagent racks 90 and 92, containing buffers for enrichment and clean-up, respectively. In the center deck 94 of module 88 is a thermomixer 96 in which enrichment beads are prepared and hybridized to templated beads. The module also contains a dia-filtration column designed to purify templated beads while being continuously agitated. A syringe pump and valve 100 are located behind a door 102 on the right side of module 88, above a shield 98 and above an inline filter 104 that is used to concentrate and buffer-exchange the beads in the process. In some embodiments, the entire process can be monitored through clear panel doors. The module can be configured to carry out each of steps 52-62 shown in FIG. 2.

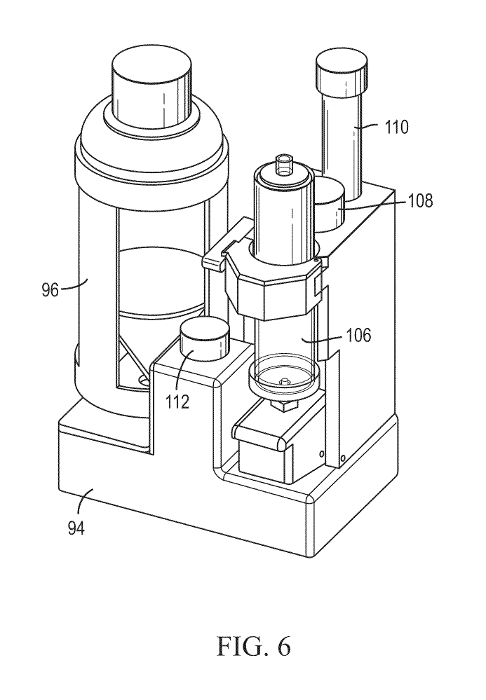

FIG. 6 is an enlarged view of center deck 94 of the enrichment module 88 shown in FIG. 5, and shows an enricher column 106, a container 108 for enrichment beads, a release tube 110, and a hybrid tube 112. In some embodiments, enrichment column 106 can comprise a mechanical device and method for bead enrichment that does not rely of surface chemistry, has very low or no non-specific adsorption, and is amenable to automation.

In some embodiments, a track-etched filtration membrane or any filtration partition that comprises straight-through holes can be used. Filtration membranes with straight-through holes or pores prepared with polymeric materials are readily available commercially with pore size ranging from 0.1 μm to 10 μm. The polymeric materials can comprise, for example, poly(tetrafluoroethylene), polycarbonate, polyacrylic, or the like. The surfaces of these polymer membranes can be rendered hydrophilic, for example, by coating or surface grafting with a hydrophilic polymer. Membranes with straight-through holes or pores can also be prepared with silicon wafers or metal thin films, for example, but not limited to, nickel and nickel alloys. The straight-through holes or pores can have narrow size distribution in the range of 2-10% CV. A track-etched polycarbonate filter membrane with straight-through holes of 0.4 pin can be used and can retain micro-spheres of 0.42 μm size.

In some embodiments, the outlet and inlet of the filtration device can be connected to fluidic devices or valves. In some embodiments, the inlet and outlet can be independently connected to reservoirs containing mobile phases, for example, aqueous electrolytes, buffers or de-hybridization buffers.

In some embodiments, a filtration membrane can be used, for example, a mesh or a stainless steel mesh. The filtration membrane can be bonded to a gasket or a gasket can otherwise be used to seal the enrichment column, in some embodiments.

In some embodiments, the present teachings provide researchers with a cost-effective sequencing solution with unprecedented accuracy.

In some embodiments, in-line filters are used to non-magnetically concentrate beads and perform buffer exchanges. A dia-filtration process can be used in lieu of the manual glycerol cushion and centrifugation. Instead of sonication, beads can be de-aggregated using sheer flow through a syringe valve. These features can enable greater scalability and ease of use.

In some embodiments, a sequencing system is provided that exhibits increased sequencing throughput by several orders of magnitude over gel based systems and can be instrumental in improving understanding of genomics and human disease. In some embodiments, the present teachings give end-users the most cost-effective sequencing platform on the market.

It is to be understood that each of the publications referenced herein is independently incorporated herein in its entirety by reference.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, as illustrated by the range of from 1 to 5.

It is noted that, as used in this specification and the appended claims, the singular forms “a,” “an,” and “the,” include plural referents unless expressly and unequivocally limited to one referent. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the devices, systems, and methods of the present disclosure without departing from the scope its teachings. Other embodiments of the disclosure will be apparent to those skilled in the art from consideration of the specification and practice of the teachings disclosed herein. It is intended that the specification and examples be considered exemplary only.

What is claimed is:

1. A method of enriching templated beads from a mixture of templated beads and non-templated beads, the method comprising:
providing a mixture of templated beads and non-templated beads;
combining the mixture with a plurality of enrichment beads;
binding one or more of the enrichment beads with one or more of the templated beads to form one or more respective capture complexes;
separating the one or more capture complexes from the non-templated beads to form one or more separated capture complexes; and
recovering the one or more templated beads from the one or more separated capture complexes by separating the one or more templated beads from the one or more enrichment beads, to form one or more recovered templated beads.

2. The method of claim 1, wherein the binding comprises hybridizing the one or more enrichment beads to one or more respective templated beads.

3. The method of claim 1, further comprising forming the templated beads in an emulsion PCR reaction.

4. The method of claim 1, wherein the templated beads comprise PCR-amplicon bearing microspheres.

5. The method of claim 1, wherein separating comprises using a size-exclusion filtration material.

6. The method of claim 1, wherein the separating comprises depth-filtration separation.

7. The method of claim 1, wherein the recovering comprises eluting one or more of the templated beads from the one or more separated capture complexes.

8. The method of claim 7, wherein the recovering comprises passing the templated beads through the filter and retaining the enrichment beads.

9. The method of claim 1, wherein each of the templated beads and each of the non-templated beads has a diameter of from 0.25 μm to 1.2 μm.

10. The method of claim 1, wherein each of the templated beads and each of the non-templated beads has a diameter of from 0.5 μm to 1.0 μm.

11. The method of claim 1, wherein the one or more enrichment beads each has a diameter of from 2.0 μm to 20.0 μm.

12. The method of claim 1, wherein the one or more enrichment beads have an average diameter of from 6.4 μm to 6.8 μm.

13. The method of claim 1, further comprising denaturing a template or amplicon tethered to the one or more recovered templated bead.

14. The method of claim 1, wherein the templated beads and the non-templated beads each comprise a metal material and the method further comprises magnetically manipulating the recovered templated beads.

15. A system for enrichment of templated beads from a mixture of templated beads and non-templated beads, the system comprising:
a mixture of templated beads and non-templated beads having a first average diameter;
a plurality of enrichment beads having a second diameter, each enrichment bead being functionalized to bind with one or more of the templated beads to form one or more respective capture complexes; and
a separation device comprising a size-exclusion filtration material having an average pore size, wherein the average pore size is larger than the first average diameter and smaller than the second diameter.

16. The system of claim 15, wherein the filtration material comprises a hydrophobic material.

17. The system of claim 15, wherein the filtration material comprises a polypropylene material.

18. The system of claim 15, wherein templated beads comprise PCR-amplicon bearing microspheres.

19. The system of claim 18, wherein the PCR-amplicon bearing microspheres comprise respective monoclonal populations of amplicons.

20. The system of claim 15, further comprising:
one or more buffer solutions disposed in one or more pre-filled containers;
a thermomixer configured to prepare enrichment beads; and
a dia-filtration column configured to purify and agitate templated beads.
